(19) United States
(12) Patent Application Publication
(10) Pub. No.: US 2015/0201910 A1
(43) Pub. Date: Jul. 23, 2015

(54) 2D-3D RIGID REGISTRATION METHOD TO COMPENSATE FOR ORGAN MOTION DURING AN INTERVENTIONAL PROCEDURE

(71) Applicant: CENTRE FOR IMAGING TECHNOLOGY COMMERCIALIZATION (CIMTEC), London (CA)

(72) Inventors: Shuang-ren ZHAO, London (CA); Aaron Fenster, London (CA); Tharindu De Silva, London (CA); Aaron D. Ward, London (CA)

(73) Assignee: CENTRE FOR IMAGING TECHNOLOGY COMMERCIALIZATION (CIMTEC), London (CA)

(21) Appl. No.: 14/158,407

(22) Filed: Jan. 17, 2014

(51) Int. Cl. A61B 8/08; G01S 7/52; A61B 10/04; A61B 8/12; A61B 8/00; G06T 7/00

(52) U.S. Cl. CPC A61B 8/5253; A61B 8/463; A61B 8/5207; A61B 8/466; G06T 7/0036; A61B 8/0841; A61B 10/04; A61B 8/12; A61B 8/483; G01S 7/52053

(57) ABSTRACT

Disclosed herein is a system and method for generating a motion-corrected 2D image of a target. The method comprises acquiring a 3D static image of the target before an interventional procedure. During the procedure, a number of 2D real time images of the target are acquired and displayed. A slice of the 3D static image is acquired and registered with one 2D real time image and then the location of the 3D static image is corrected to be in synchrony with body motion. The motion corrected 2D image of the target is then displayed.

### TECHNICAL FIELD

The present relates to ultrasound imaging techniques, and more particularly to an image-based registration algorithm for 2D to 3D rigid/affine ultrasound image registration technique.

### BACKGROUND

Prostate cancer is the second most frequently diagnosed cancer among men in North America [1], with prostate biopsy as the clinical standard for diagnosis. During biopsy, the physician systematically obtains approximately a dozen tissue samples from different regions of the prostate to assess disease status via histopathology analysis of the extracted tissue. Prostate biopsy is usually performed under two-dimensional (2D) trans-rectal ultrasound (TRUS) guidance by inserting a needle through the patient's rectal wall. However, with the small size of the biopsy cores taken, the presence of small, multi-focal cancers might result in negative biopsies. In fact, the false negative rate of the 2D TRUS-guided biopsy procedure is reported to be as high as 30% [3]. Poor visibility of cancer in TRUS images and the limited anatomical context available in the 2D TRUS plane make it challenging for the physician to accurately guide needles to suspicious locations within the prostate. With the aim of improving the cancer detection rate, systems have been developed [4, 5] that can plan and record biopsy locations in a 3D TRUS image acquired at the beginning of the biopsy procedure. Target biopsy locations can be identified in the 3D TRUS image with the assistance of a magnetic resonance (MR) image acquired prior to the biopsy session, in which focal lesions are more visible. The 3D TRUS image can then act as a baseline image, to guide the physician to the target biopsy locations by augmenting the 2D TRUS planes acquired during biopsy with 3D contextual information.

Although early reports of 3D TRUS guided systems are promising, some limitations have been identified that require attention [6]. Patient motion and ultrasound probe pressure can cause the prostate to move and deform during the biopsy procedure. This may lead to a misalignment between the targets identified in the initially-acquired 3D image and their corresponding locations within the patient's prostate as depicted by the real-time 2D TRUS images acquired throughout the biopsy procedure. Compensating for the prostate motion and deformation by registering the pre-acquired 3D image to the live 2D images acquired throughout the procedure is an important step toward improving the targeting accuracy of a biopsy system.

Previous approaches to compensation for prostate motion during biopsy have involved mechanical stabilization of the ultrasound probe, 3D tracking of the probe, and the use of biplanar or 3D transducers to continuously acquire richer image information supporting software-based motion compensation algorithms [4, 5, 7, 8]. The mechanically assisted 3D TRUS-guided biopsy system developed in our laboratory and described in detail in [4], uses a passive mechanical arm to track the position and orientation of the ultrasound probe during the biopsy procedure. The design yields a remote centre of motion positioned at the centre of the ultrasound probe tip that provides enhanced stability to the TRUS probe minimizing prostate motion. Several methods have been proposed in similar 3D TRUS-guided biopsy systems to register real-time TRUS images during the procedure to an initially acquired 3D image [5]. The 3D TRUS-guided biopsy system presented in Xu et al. [5] uses a magnetic tracking method to locate the ultrasound plane and it then performs an intermittent rigid registration to compensate for out-of-plane prostate motion; the registration is invoked when misalignment is detected visually by an operator. The magnetic tracker transform provides an initialization for the 2D TRUS plane within the world coordinate system in their system. In that work, however, registration accuracy was measured with a phantom study. Baumann et al, [7] presented a method relying on the simultaneous real-time acquisition of dual, orthogonal 2D TRUS images acquired from a 3D ultrasound probe. The same authors presented an algorithm [8] to compensate for motion using 3D TRUS volumes acquired continuously throughout the biopsy session. This system does not use any method to track ultrasound probe motion; therefore, it relies only on the image information for tracking and uses a coarse-to-fine image-based approach to limit the search space during optimization. In addition, this approach requires a special 3D ultrasound probe with enhanced functionality that could simultaneously acquire orthogonal 2D TRUS planes, and image acquisition occurs at a lower frame rate, compared to more conventional 2D TRUS, Moreover, compared to 2D TRUS images, orthogonal 2D planes deliver considerably more spatial information; registration of a single 2D TRUS plane to a 3D TRUS image is a more challenging problem.

To the best of our knowledge, no previous work has described and evaluated on human clinical images a method for the registration of 2D TRUS to 3D TRUS images for prostate motion compensation during biopsy. Such a technique, if properly validated, will make it possible to perform prostate motion compensation on 3D biopsy guidance systems that use readily available 2D ultrasound probes for live image acquisition throughout the procedure, permitting more widespread use of targeted biopsy systems and thus greater potential impact on the patient population. 2D-3D registration methods have been applied to several other interventional applications in image-guided procedures (see Markelj et al, [9]). Birkfellner et al. [10] compared the performance of several image similarity measures and optimization techniques for 2D-3D registration of fluoroscopic images and found that cross-correlation is an optimal metric for intra-modality matching. Wein et al. [11] presented a method to compensate for respiratory motion during abdominal biopsies and ablations under ultrasound guidance, optimizing local normalized cross-correlation using the Powell-Brent direction search technique. Although these previous successes speak to the potential feasibility of addressing the issue of prostate motion compensation in software using a 2D-3D intensity-based image registration technique, prostate appearance on TRUS and motion characteristics during biopsy may differ from those of other organs due to different tissue stiffness properties and flexibility of surrounding anatomical structures.

A number of methods for compensating for respiratory motion during image-guided interventional procedures are known in the art. Among these are included breath-hold methods, gating methods (published US patent application no. 2012/0230556), and real-time tracking methods (U.S. Pat. No. 8,155,729).

Another approach taught in the art is for estimating motion of an organ and then transformation of the image as taught by published US patent application no. 2008/0246776. A further approach is to incorporate a mode of respiratory motion into the registration to compensate for the respiratory motion and registration of pre-operative volumetric image dataset with the intraoperative image as disclosed in published US patent application no. 2010/0310140.

Methods of computing a transformation linking two images is known in the art as taught by U.S. Pat. No. 6,950,542. Methods of registration of images are also known. U.S. Pat. Nos. 7,912,259 and 7,616,836 teach the use of multiple feature masks motion compensation between first and second images in a temporal sequence and deriving a displacement between two images is known. In particular, 2D-3D registration methods that compensate for organ motion have been applied to several other interventional applications in image-guided procedures. Among these is registration of 2D images with a 3D reference image as disclosed in U.S. Pat. No. 8,317,705. Also, Wein et al. developed a method of acquiring a pre-procedure ultrasound sweep over a whole liver during a breath-hold. This data serves as reference 3D information. The real-time ultrasound image is tracked and a position sensor attached to the patients skin is employed to detect movement due to breathing motion of a target within the liver. Respiratory motion can be compensated using a slice-to-volume registration approach. The method optimizes local normalized cross correlation (NCC) using the Powell-Brent direction search technique.

A number of research groups and companies are working on developing solutions for compensating for respiratory motion during image-guided interventional procedures, including breath-hold methods, gating methods, and real-time tracking methods. Breath-hold and gating techniques have the disadvantage of increasing treatment time and can be uncomfortable for patients.

One known approach that is being used for radiotherapeutic treatment of lung cancer involves using respiratory gating to compensate for motion. The method involves tracking tumor motion/location in x-ray images by using a robot-mounted linear accelerator (Accuray Cyberknife).

Another current approach that has been developed for motion compensation is to track ultrasound transducers and/or magnetically tracking needle tips (Traxtal Inc., CAS Innovations AG, etc.). This system involves alignment of pre-operative CT or MRI images during the breathing phase of pre-operative CT or MRI images.

Research groups have also proposed creating pre-operative models of the liver motion from 4D MRI acquisitions for a patient and registering the model to tracked 2D ultrasound images, using PCA based methods. However, this approach is expensive, time-consuming and cannot reproduce breathing irregularities which vary from patient to patient.

2D-3D registration methods that compensate for organ motion have been applied to several other interventional applications in image-guided procedures [see Markelj et al. paper]. Please refer to De Silva et al. paper. The respiratory motion can be compensated for “using a slice-to-volume registration approach” and “optimizes local normalized cross correlation (NCC) using the Powell-Brent direction search technique”.

3D TRUS-guided systems have been developed to improve targeting accuracy during prostate biopsy. However, prostate motion during the procedure is a potential source of error that can cause target misalignments.

### BRIEF SUMMARY

We have developed a new and non-obvious 2D-3D intensity-based and geometric based image registration technique to compensate for prostate motion with sufficient accuracy and speed to be translated to clinical use for 3D biopsy guidance. Our method is applicable to both prostate, liver and other organs/tissues and includes features that increase the speed of the algorithm. In addition to medical applications, motion compensation has applicability to fields requiring the ability to detect and track moving objects/targets, including machine vision (i.e. dynamic image analysis, object and environment modeling), military, sensor (i.e. object tracking), and mining.

Accordingly, there is provided a method for generating a motion-corrected 2D image of a target, the method comprising:

acquiring a 3D static image of the target before an imaging procedure;

during the procedure, acquiring and displaying a plurality of 2D real time images of the target;

acquiring one slice of the 3D static image and registering it with at least one 2D real time image;

correcting the location of the 3D static image to be in synchrony with a reference parameter; and

displaying the reference parameter corrected 2D image of the target.

In one example, the method includes: displaying 2D real time images as an ultrasound video stream collected at a video frame rate of up to 30 frames per second.

In one example, the method further comprising: matching and minimizing target goals or metric values for the 2D real time images.

In another example, the method described above, in which the 2D-3D registration is rigid/affine. Local optimization searches the minimized value which mature of a 2D slice inside a 3D volume image. Global optimization searches the minimized value which mature of a 2D slice inside a 3D volume image. Estimated values are estimated from a few prior output parameters of the successful 2D-3D image registrations and the priori from last period of respiration. The estimation can be a polynomial or Fourier series.

In another example, the method, described above, in which one slice of the 3D static image is matched to the correct plane as the 2D real time image.

In another example, the method, described above, in which the reference parameter is body movement. The 2D real time image is matched according to the body movement.

In another example, the method, described above, the registering of the 2D and 3D images are done visually.

In another example, the method, described above, the registering of the 2D and 3D images are done by identifying corresponding points in the 2D and 3D images and finding the best translation/rotation/shearing transform to achieve approximate registration.

In another example, the method, described above, for each 2D real time image:

determining the corresponding plane in the 3D static image; and

finding the corresponding 2D real time images in the 3D static image volume to determine which slice therein matches the 2D real time image.

In another example, the method, described above, further comprising:

minimizing errors or metric values in registering of the 2D and 3D images by applying a local optimization method.

In another example, the method, described above, further comprising:
minimizing the errors or metric values in registering of the 2D and 3D images by applying Powell's optimization algorithm.

In another example, the method, described above-further comprising:

minimizing the errors or metric values in registering of the 2D and 3D images by applying particle swarm optimization to calculate degree of matching between the 2D and 3D images. Powell's optimization algorithm minimizes registration error measurement by calculating the target registration error (TRE). Powell's optimization algorithm minimizes registration error measurement by calculating the metric value using manually identified fiducials in the target.

In another example, the method, described above, the multiple initial parameters for 2D-3D image registration include the output parameters of the prior 2D-3D registration; the estimated output parameters using a group of the prior 2D-3D registration; or the output parameter of 2D-3D registration from last period of respiration. The particle swarm optimization increases the registration speed when matching large high-resolution 2D and 3D images comparing with other global optimization method. Powell's optimization algorithm or the particle swarm optimization is continuously applied throughout the procedure by acquiring and registering the 2D real time images every 30-100 millisecond.

In another example, the method, described above, if the local optimization method fails, a global optimization method is applied, the global optimization method being particle swarm optimization method. The registration is carried out as a background process to continuously compensate for motion during the procedure.

In another example, the method, described above, a graphics processing unit (GPU)-accelerates the registration.

In another example, the method, described above, the target is the liver.

In another example, the method, described above, the target is the prostate gland.

In another example, the method, described above, the 2D and 3D images are TRUS images.

In another example, the method, described above, the imaging procedure is an interventional procedure. The interventional procedure is a biopsy procedure.

In another example, the method, described above, the imaging procedure is remote sensing (cartography updating),

In another example, the method, described above, the imaging procedure is astrophotography,

In another example, the method, described above, the imaging procedure is computer vision in which images must be aligned for quantitative analysis or qualitative comparison.

In another example, the method, described above, in which the 2D-3D registration is non-rigid.

According to another aspect, there is provided a method for generating a motion-corrected 2D image of a target, the method comprising:

acquiring a 3D static image of the target before an interventional procedure;

during the procedure, acquiring and displaying a plurality of 2D real time images of the target;

acquiring one slice of the 3D static image and registering it with at least one 2D real time image;

correcting the location of the 3D static image to be in synchrony with body motion; and

displaying the motion corrected 2D image of the target.

According to another aspect, there is provided a system for generating a motion-corrected 2D image, the system comprising:

an ultrasound probe for acquiring data from a target during an interventional procedure;

an imaging device connected to the ultrasound probe for displaying data acquired by the ultrasound probe;

a computer readable storage medium connected to the ultrasound probe, the computer readable storage medium having a non-transient memory in which is stored a set of instructions which when executed by a computer cause the computer to:
acquire a 3D static image of the target before the procedure;
during the procedure, acquire and display a plurality of 2D real time images of the target;

acquire one slice of the 3D static image and register it with at least one 2D real time image;

correct the location of the 3D static image to be in synchrony with body motion; and

display the motion corrected 2D image of the target.

According to yet another aspect, there is provided a system for generating a motion-corrected 2D image, the system comprising:

a probe for acquiring data from a target during an imaging procedure;
an imaging device connected to the probe for displaying data acquired by the probe;

a computer readable storage medium connected to the probe, the computer readable storage medium having a non-transient memory in which is stored a set of instructions which when executed by a computer cause the computer to:
acquire a 3D static image of the target before the procedure;
during the procedure, acquire and display a plurality of 2D real time images of the target;

acquire one slice of the 3D static image and register it with at least one 2D real time image;

correct the location of the 3D static image to be in synchrony with a reference parameter; and

display the reference parameter corrected 2D image of the target.

### BRIEF DESCRIPTION OF THE DRAWINGS

In order that the discovery may be readily understood, embodiments of the invention are illustrated by way of example in the accompanying drawings.

FIG. 1 is a flow diagram showing 2D-3D registration workflow. FIG. 1(a) The outside connections of 2D-3D registration workflow. FIG. (b) The inside of 2D-3D registration workflow.

FIG. 2(a), 2(b), 2(c) are histograms of TRE before and after registration for prostate biopsy protocol data. FIG. 2(a) shows before registration; FIG. 2(b) shows after registration; and FIG. 2(c) showing after continuous registration after every second;

FIG. 3 is a histogram showing TRE before registration, after registration and after continuous registration every second for each biopsy in biopsy prostate protocol;

FIG. 4 are images before and after registration. The Left column illustrates real-time 2D TRUS images; the Middle column illustrates corresponding images before registration assuming to prostate motion (from the transformation given by the mechanical tracking system); and the right column illustrates corresponding images after registration;

FIG. 5(a), 5(b), 5(c) are graphs showing TRE as a function of time elapsed from the start of the biopsy. FIG. (a) TRE before registration; FIG. (b) TRE after registration; and FIG. (c) TRE after registering the images acquired every second;

FIG. 6(a), 6(b), 6(C) are histograms for TRE before and after registration for probe pressure protocol data. FIG. 6(a) shows TRE distribution before registration; FIG. 6(b) shows TRE distribution after registration; and FIG. 6(c) shows TRE distribution with the best rigid alignment for the identified fiducials;

FIG. 7 is a graph showing TRE as a function of metric value during the optimization. Initial points (circles), converged (squares) and converging points (crosses);

FIG. 8 is a graph showing TRE distributions before registration, during convergence and after registration;

FIG. 9 are graphs showing mean and standard deviations of normalized cross-correlation values for 16 image pairs of eight patients in the six-degrees-of-freedom transformation space, one degree-of-freedom varying at a time. The zero location in the x-axis corresponds to real-time 2D-TRUS frame;

FIG. 10 are graphs showing normalized cross-correlation values for a single image pair of a biopsy for 3 patients (each biopsy represented by a separate line pattern) in the six-degrees-of-freedom transformation space, one degree-of-freedom varying at a time. The zero location in the x-axis corresponds to real-time 2D-TRUS frame; and

FIG. 11 is a graph showing TRE as a function of distance to the probe tip.

Further details of the discovery and its advantages will be apparent from the detailed description included below.

### DETAILED DESCRIPTION

Ultrasound is a widely used imaging modality that is traditionally 2D. 2D ultrasound images remove 3D volume and three-dimensional information that allows for determining shapes, distances, and orientations. Ultrasound is used in medical, military, sensor, and mining applications

In interventional oncology, ultrasound is the preferred intra-operative image modality for procedures, including biopsies and thermal/focal ablation therapies in liver & kidney, laparoscopic liver surgery, prostate biopsy and therapy, percutaneous liver ablation, all other abdominal organs and ophthalmic intervention known to those skilled in the art. Some brain interventions also use ultrasound, although MR and CT are more common.

Ultrasound allows for “live information” about anatomical changes to be obtained with the requirement for further radiation dose to patient or physician. For image-guided interventions, it can be difficult for a surgeon to navigate surgical instruments if the target organ is moving either due to patient motion (i.e. breathing and cardiac motion the patient respiratory motion) or ultrasound probe pressure (causing movement and deformity of the organ), in any procedure that requires a needle or needles, particularly in ultrasound-guided interventional procedures, it is important to be able to correct for motion of an organ thereby allowing the interventionist to be able to track and position/align needles relative to the planned trajectory, nearby vulnerable structures and to position them at their target position with a high degree of precision and accuracy. To gain acceptance in the clinical practice the speed of registration must be both accurate and fast.

2D/3D registration is a special case of medical image registration which is of particular interest to surgeons. 2D/3D image registration has many potential applications in clinical diagnosis, including diagnosis of cardiac, retinal, pelvic, renal, abdomen, liver, and tissue disorders. Applications of 2D/3D registration also has applications in radiotherapy planning and treatment verification, spinal surgery, hip replacement, neurointerventions, and aortic stenting.

Target organ motion during a procedure can cause target misalignments between the initially acquired 3D image and their corresponding locations within the patient's prostate or liver as depicted by the real-time 2D ultrasound images acquired. Although our method was developed and tested for prostate gland and liver applications, it is applicable to all organs where motion compensation is required.

Accurate and fast registration to compensate for motion during minimally invasive interventions, such as a biopsy, is an important step to improve the accuracy in delivering needles to target locations within any organ.

The method steps described herein are embodied in a computer readable storage medium which includes a non-transient memory with a computer program stored thereon. The computer program represents a set of instructions to be executed by a computer. The computer readable storage medium is connected to the ultrasound probe and when required cause the computer to carry out those method steps described herein: In addition to a biopsy procedure the methods described herein can also be applied to other non-limiting interventional procedures such as image-guided interventional procedures including ablations and laparoscopies and the like.

As used herein the term “imaging procedure” is intended to mean a computerized technique or procedure such as ultrasonography, computed tomography, magnetic resonance imaging, positron emission tomography, single-photon emission computed tomography that generates a visual representation of an object.

As used herein, the term “reference image” is intended to mean an image which is typically a first image, or any image that is designated as the reference to which other images are referenced. A reference image can be any of the following: 3D MRI image, 3D CT image, 3D PET image, 3D SPECT image, and 3D ultrasound image.

As used herein, the term “reference parameter” is intended to mean body or organ/tissue movement or motion, or any other object motion. Specifically, reference parameter means a value generated by the registration process that described the “goodness” of the registration. A typical parameter is the Normalized Cross Correlation or Mutual Information.

As used herein, there term “normalized cross correlation” is intended to mean a group of metrics including normalized cross correlation metric, Kullback-Leibler distance metric, Normalized Mutual Information Metric, Mean Squares Histogram Metric, Cardinality Match Metric, Kappa Statistics Metric, and Gradient Difference Metric.

### A. Geometric-Based 2D-3D Rigid/Affine Registration

We have developed an image-based registration algorithm for 2D to 3D rigid/affine ultrasound image registration technique to compensate for organ motion as a surgical instrument, such as a biopsy needle, is inserted into a target organ, for example. liver, and prostate gland. Specifically, this algorithm was developed for compensating for liver motion, but can be applied to other organs. The algorithm allows for tracking of surgical instruments in real-time. Speed is important for there to be any clinical use.

To perform 2D/3D registration, the 3D and 2D data are brought into dimensional correspondence (geometrically aligned). Registration algorithms compute transformations to set correspondence between the 2D image and one slide of the 3D image. The slice is chosen arbitrarily. Our registration algorithm computes transformations of 3D data into 2D with a particular application to prostate and liver, however it can be applied to other interventional applications in image-guided procedures including other organs i.e. lung, venter, breast and other fields, for example recovering the 2D tracker information and reconstruction 3D image from 2D frames without tracker information. Here the tracker information are transform parameters like rotation angles and translatory distance of the 2D image tracker system. 2D/3D registration also has applications in fields in addition to medical such as machine vision (i.e. dynamic image analysis, object and environment modeling), military, sensor (i.e. object tracking), and mining.

As the biopsy needle is inserted into a target organ, and if the patient is breathing, then the target organ will be moving. There can also be non-rigid deformation due to ultrasound probe pressure on the organ. The algorithm allows for the identification of the target on another imaging modality. The aim of the algorithm is to correct the location of the 3D image so that it is in synchrony with those 2D ultrasound images acquired during the procedure. That is the 3D image must be in synchrony with body motion.

The method for generating a motion-corrected 2D image uses a combination of geometric-based 2D-3D rigid registration and intensity-based 2D-3D rigid registration.

During the biopsy procedure, a 2D real-time ultrasound video stream, which includes intra-procedural 2D real-time live images of the target organ or tissue, is acquired and displayed on a computer screen (an imaging device). Typically, the acquisition and display of the video stream is done at a video frame rate of up to 30 frames per second. The target organ or tissue is typically one that is suspected of being diseased or is known to be diseased. A pre-procedural/interventional target image (a 3D static image) with the target identified, such as a tumor, is acquired at the beginning of the procedure before the interventionist inserts the needle into the target organ. The data sets to be registered are defined in coordinate systems.

An acquired 2D image is compared with one slice in the 3D image to determine if they match. This is done as a precaution in case the parameter of transform changed. A target goal is set up, and if the goals are well matched, then the function value of the. target goal will be minimized. Examples of the transform's parameters include rotation, translation, shearing and scaling. The goal here is to find the best slice inside the 3D image. The best slice is defined by the transform parameters, which looks like the 2D image.

The initialization phase for the algorithm involves correcting the location of the 3D image so that it is in synchrony with body motion, caused by breathing, heart beat and the like, of that viewed with 2D ultrasound images acquired during the procedure. For each 2D image taken, the corresponding plane in the 3D volume must be found. The 3D image can be moved to the correct plane as the 2D image. Usually the 2D image is moved according to patient movement, such as breathing. At this point, the user needs to determine which slice in the 3D image matches the live image, i.e. the user must find the corresponding 2D image in the pre-acquired 3D volume, which can be problematic. We have successfully addressed this problem by using a geometric-based 2D-3D registration algorithm. It was done by taking a 3D image and extracting the 2D image from it. That is, a 2D slice (2D extracted image) is taken out of the 3D volume image. The 3D image and 2D extracted image are approximately lined up to recover a 3D contextual form of an image (also known as correct geometrical context). This means the 2D and 3D images appear to be aligned. The alignment can be done either visually or by using an algorithm such as by identifying corresponding points in the 2D and 3D images and finding the best translation/rotation/shearing transform to achieve approximate registration. The resulting image is a 3D image that can be looked at in the same plane as the 2D real-time image.

The 3D image (2D extracted image) is compared to a 2D real-time image. If the two images do not match exactly, the plane where the 3D image and the 2D image do not match exactly is extracted. During registration, an image similarity metric is optimized over a 3D transformation space. Accurate definition of similarity measure is a key component in image registration. To do this, minimizations for registration are performed. Motion is extracted in 12 degrees of freedom or less and then the plane is moved and motion is extracted in different angles using different image similarity metrics, including normalized cross-correlation, versor rigid transform or affine transform. Powell's optimization or particle swarm optimization is applied to calculate degree of matching. Powell's method is used to minimize registration error measurement. It is used for local minimizations (i.e. will only find a local solution). There is no guarantee that a correct match will be found applying only Powell's method.

In order to increase the success of the registration a few multiple initial parameters are applied, which for example are (a) the output parameters of prior 2D-3D registration results; (b) estimated parameters using a few groups output parameters of prior 2D-3D registration; (c) the output parameters obtained in the same time of last respiration; and (d) the output parameter of the first successful 2D-3D registration.

The re-initialization phase. As described above-Powell's method optimization is a local minimization, which can fail. The particle swarm optimization can be carried out to find the global solution in case Powell's method fails. Using the particle swarm optimization increases the global optimization speed. It can be calculated parallel for all particles. The initial parameters for the optimization of particle swarm method are the same as those with the Powell method. If the time of the calculation for particle swarm method takes too long, the estimated initial parameters will be used for this 2D frame.

Estimation of the initial parameters for of the 2D-3D registration for each 2D frame. Before the calculation of 2D-3D image registration, the initial transform parameters are estimated from the changes of known parameters y(tₖ) has been calculated for a few (N) prior frames. The estimation is done through polynomial series or Fourier series.

$$f ( {a}_{i} , t ) = {\sum }_{i = 0}^{I - 1} {a}_{i} {t}^{i} , \\ \{ {a}_{i} \} = arg min {\sum }_{i = - N}^{- 1} {w}_{n} \vert {y}_{n} - f ( {a}_{i} , {t}_{n} ) \vert , \\ f ( {b}_{i} , t ) = {\sum }_{i = 0}^{I - 1} {b}_{i} exp ( \sqrt{- 1} \frac{2 π}{τ} it ) , \\ \{ {b}_{i} \} = arg min {\sum }_{n = - N}^{- 1} {w}_{n} \vert {y}_{n} - f ( {b}_{i} , {t}_{n} ) \vert ,$$

ƒ(aᵢ,t) or ƒ(bᵢ,t) is one of estimated parameter. wₖ is the weight which can be different with k. T is the period of the respiration. yₖ is one of known registration parameter in recorded in different time tₖ of respiration. N is the number of 2D frames in a period of the respiration, l is the number of coefficient bᵢ. t=0 is corresponding to current time in which the 2D-3D registration is perfumed. We assume the parameters are changed independently. The estimated initial parameter is

ƒ(bᵢ,t)|ₜ₌₀,ƒ(bᵢ,t)|ₜ₌₁, . . .

or

ƒ(aᵢ,t)|ₜ₌₀,ƒ(aᵢ,t)|ₜ₌₁ . . .

At this point finding the best match quickly is important for the user. Once the user has an estimation of normalized cross-correlation, the 3D image (target) is transformed to the current location as that obtained from the 2D real-time image. The 3D image is transformed to achieve the best possible correspondence with the 2D image(s). The transformation is 2D/3D image-to-image registration.

The algorithm processes up to 30 frames per second. A graphics processing unit (GPU)-based implementation was used to improve the registration speed to register from 2D to 3D. The speed of registration must be fast to gain acceptance in the clinical practice.

### Affine Transformation

The affine transformation has 12 parameters which as following form, see Eq. (8.11) of ref[20] below:

$$[ \begin{matrix}{x}^{′} \\ {y}^{′} \\ {z}^{′}\end{matrix} ] = [ \begin{matrix}{M}_{00} & {M}_{01} & {M}_{02} \\ {M}_{10} & {M}_{11} & {M}_{12} \\ {M}_{20} & {M}_{21} & {M}_{22}\end{matrix} ] [ \begin{matrix}x - {C}_{x} \\ y - {C}_{y} \\ z - {C}_{z}\end{matrix} ] + [ \begin{matrix}{T}_{x} + {C}_{x} \\ {T}_{y} + {C}_{y} \\ {T}_{z} + {C}_{z}\end{matrix} ]$$

Coordinates after affine transformation:

$$[ \begin{matrix}{x}^{′} \\ {y}^{′} \\ {z}^{′}\end{matrix} ]$$

Coordinates before transformation:

$$[ \begin{matrix}x \\ y \\ z\end{matrix} ]$$

The original point can be any point in the space. The center of the rotation is at

$$[ \begin{matrix}{C}_{x} \\ {C}_{y} \\ {C}_{z}\end{matrix} ]$$

There are two kind of parameters for this transformation which are in the following:

1) Affine matrix parameter:

$$[ \begin{matrix}{M}_{00} & {M}_{01} & {M}_{02} \\ {M}_{10} & {M}_{11} & {M}_{12} \\ {M}_{20} & {M}_{21} & {M}_{22}\end{matrix} ] and$$

2) Translation parameters:

$$[ \begin{matrix}{T}_{x} \\ {T}_{y} \\ {T}_{z}\end{matrix} ]$$

### Particle Swarm Optimization

For particle swarm optimization[21][22] we assume we have J parameters: x={x₀, . . . , xⱼ, . . . , xⱼ₋₁}. The optimization problem is to find optimal x with the function ƒ(x), i.e.

x=arc minƒ(x)

Assume there are N particles. The positions are of i-th particle are

xᵢ={xᵢ₀, . . . ,xᵢⱼ, . . . x_{if-1}}

Assume k is iteration index. The position of particles are updated according to

xᵢ^{k+1}=xᵢ^{k}+vᵢ^{k+1}

where

vᵢ^{k+1}=β[vₜ^{k}+c₁r₁(yᵢ^{k}−xᵢ)+c₂r₂(gᵢ^{k}−xᵢ^{k})

yᵢ^{k} is k th iteration, i th particles best (personal best) position in the history.

yᵢ^{k}=arc min.{ƒ(xᵢ^{l})}i=const,l=0, . . . ,k

gᵢ^{k} k th iteration, i th particle's group best (group best) position in the history.

gᵢ^{k}=arc min.{ƒ(xᵢ^{l})}iεGᵢ,l=0, . . . ,k

Gᵢ is Group of neighbor particles. For example all particle smaller than a fix distance.

r₁,r₂ is two random variables between [0,1]. c₁,c₂ are two constant around 2, β is constant.

### GPU Calculate Particle Swarm Optimization

The functions value of

{ƒ(x₀), . . . ,ƒ(xᵢ), . . . ,ƒ(x_{N})}

is calculated in parallel.

The GPU calculates the 3D to 2D image normalized cross-relation function.

### B. Intensity-Based 2D-3D Rigid Registration

We also discovered an image-based registration technique to compensate for prostate motion by registering the live 2D TRUS images acquired during the biopsy procedure to a pre-acquired 3D TRUS image. The registration must be performed both accurately and quickly in order to be useful during the clinical procedure. This technique, an intensity-based 2D-3D rigid registration algorithm, optimizes the normalized cross-correlation metric using Powell's method, The 2D TRUS images acquired during the procedure prior to biopsy gun firing were registered to the baseline 3D TRUS image acquired at the beginning of the procedure. The accuracy was measured by calculating the target registration error (TRE) using manually identified fiducials within the prostate; these fiducials were used for validation only and were not provided as inputs to the registration algorithm. We also evaluated the accuracy when the registrations were performed continuously throughout the biopsy by acquiring and registering live 2D TRUS images every second. This measured the improvement in accuracy resulting from performing the registration as a background process, continuously compensating for motion during the procedure. To further validate the method using a more challenging data set, registrations were performed using 3D TRUS images acquired by intentionally exerting different levels of ultrasound probe pressures in order to measure the performance of our algorithm when the prostate tissue was intentionally deformed. In this data set, biopsy scenarios were simulated by extracting 2D frames from the 3D TRUS images and registering them to the baseline 3D image. A GPU-based implementation was used to improve the registration speed. We also studied the correlation between NCC and TREs.

The root mean square TRE of registrations performed prior to biopsy gun firing was found to be 1.87±0.81 mm. This was an improvement over 4.75±2.62 mm before registration. When the registrations were performed every second during the biopsy, the RMS TRE was reduced to 1.63 0.51 mm. For 3D data sets acquired under different probe pressures, the RMS TRE was found to be 3.18±1.6 mm. This was an improvement from 6.89±4.1 mm before registration. With the GPU based implementation, the registrations were performed with a mean time of 1.1 s. The TRE showed a weak correlation with the similarity metric. However, we measured a generally convex shape of the metric around the ground truth, which may explain the rapid convergence of our algorithm to accurate results.

We therefore determined that registration to compensate for prostate motion during 3D TRUS-guided biopsy can be performed with a measured accuracy of less than 2 mm and a speed of 1.1 s, which is an important step towards improving the targeting accuracy of a 3D TRUS-guided biopsy system.

### Data Acquisition

We acquired images from human clinical biopsy procedures using a mechanically assisted 3D TRUS-guided biopsy system [4] in a study approved by the Human Research Ethics Board of Western University. The system, using a commercially available end-firing 5-9 MHz TRUS transducer probe (Philips Medical Systems, Seattle, Wash.), acquired a 3D TRUS image at the beginning of the biopsy procedure, and then acquired and displayed 2D TRUS images at a video frame rate (7-30 frames per second) during the biopsy session. The mechanical encoders attached to the ultrasound probe tracked its 3D position and orientation throughout the procedure, Using this system, we recorded images acquired during clinical biopsy procedures under two different protocols, in order to obtain datasets to test the robustness of the registration algorithm under different motion characteristics of the prostate. For both protocols, all 3D TRUS images were recorded prior to taking any biopsy tissue samples. For the first protocol (referred to hereinafter as the biopsy protocol), we acquired images from eight subjects. Following the standard operating procedure for 3D TRUS-guided biopsy in our trial, a 3D TRUS image was acquired at the start of the biopsy procedure, and then live 2D TRUS images were recorded at one frame per second from the sequence of images that follows at video frame rate. For the second protocol (hereinafter referred to as the probe pressure protocol), images were acquired from ten subjects. 3D TRUS images were acquired after applying three different probe pressures on the prostate gland centrally: 1) applying a medium probe pressure, similar to what a physician usually applies during a biopsy, 2) applying a low probe pressure that caused minimal prostate displacement, and 3) applying a high probe pressure that caused substantial prostate deformation and anterior displacement, This yielded a data set with prostate motions and deformations under a wide range of ultrasound probe pressures.

### 2D-3D Registration—Biopsy Protocol

For each of the eight subjects, we selected 1-3 2D TRUS images per patient 1-2 seconds prior to biopsy needle insertion. This choice of 2D TRUS images was motivated by the fact that accurate alignment of the predefined targets with the intra-procedure anatomy is chiefly required immediately prior to biopsy, when a tissue sample is to be taken from an intended biopsy target. We analyzed 16 such images from eight subjects.

The transformation, T_{Tr}:ψ→Ω given by encoders on the joints of the linkage of the mechanical biopsy system, maps each live 2D TRUS image, lₗᵢᵥₑ:ψ→ [symbol] , to the world coordinate system of the previously acquired 3D TRUS image l_{base}:Ω→ [symbol] where ψ⊂ [symbol] ² and Ω⊂ [symbol] ². Within the 3D world coordinate system, any differences in prostate position and orientation between the real-time 2D TRUS images and the initially-acquired 3D TRUS image are due to prostate motion within the patient, gross movements of the patient during the procedure, and the biopsy system's tracking errors. The accuracy of the initialization for the prostate motion registration algorithm is based in part on tracking errors of the biopsy system. In the system developed by Bax et al, [4], the accuracy in delivering a needle to a biopsy core in a phantom were found to be 1.51±0.92 mm. Registration of live 2D TRUS images to the pre-acquired 3D image compensates for both the tracking errors and errors due to prostate motion.

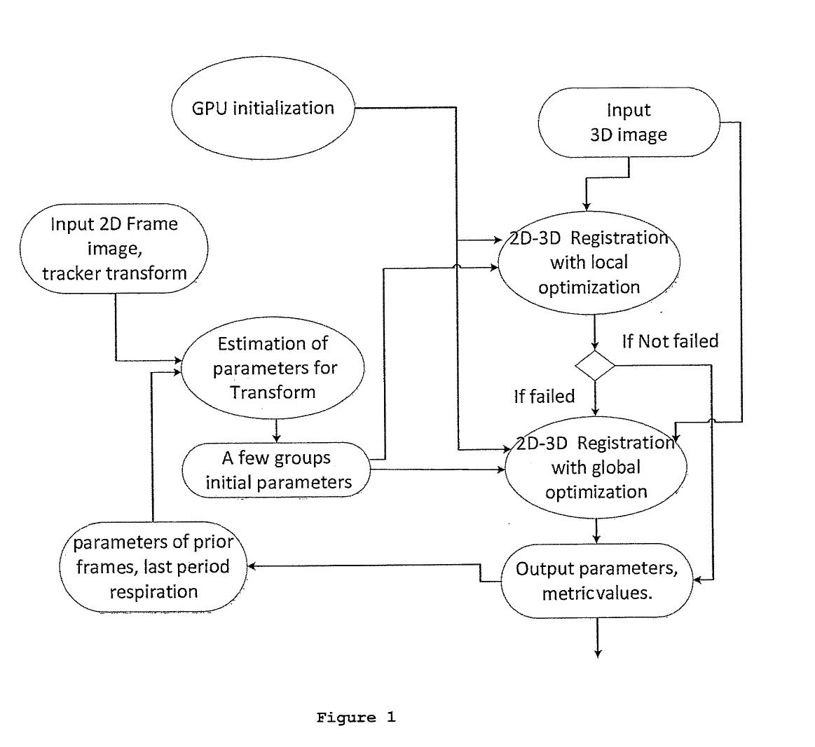

FIG. 1 illustrates the overall workflow in our method. To reduce the effects of speckle, anisotropic diffusion filtering [12](conductance parameter=2, time step=0.625) of images was used as a pre-processing step. Although there can be non-rigid deformation of the prostate due to ultrasound probe pressure [13], a rigid/affine alignment can be found with lower computational cost, so we investigated the accuracy of rigid/affine registration in this work to determine whether rigid registration is sufficient for the clinical purpose of biopsy targeting. For each 2D TRUS image, finding the corresponding plane in the pre-acquired 3D TRUS volume is a 2D-to-3D intra-modality rigid/affine registration problem. Due to limited ultrasound contrast within the prostate, reliable extraction of the boundary and other anatomic features is challenging. Therefore, we tested an intensity-based registration algorithm.

Using the mechanical tracker transform T_{Tr}, we can position and orient the 2D TRUS image lₗᵢᵥₑ within the 3D world coordinate system yielding a 3D image lₗᵢᵥₑ as follows:

lₗᵢᵥₑ(T_{Tr}(p₁))=lₗᵢᵥₑ(p₁).

where p₁ ⊂ψ.

The registration of the baseline 3D image l_{base} to lₗᵢᵥₑ is performed in this 3D world coordinate system. The objective of the registration is to find the transformation, Tᵤ:Ω→Ω, consisting of a six/twelve-parameter-vector given by u, that aligns anatomically homologous points in l_{base} and lₗᵢᵥₑ. We used normalized cross-correlation (NCC) [15] as the image similarity metric that was optimized during the registration. For two images I₁ and I₂, we optimized the objective function defined as:

$$F = arg {max}_{u} NCC ( {I}_{1} , {I}_{2} ; u ) , where \\ NCC ( {I}_{1} , {I}_{2} ; u ) = \frac{{\sum }_{p ∈ {Ω}_{1 , 2}^{{T}_{u}}} ( {I}_{1} ( p ) - {I}_{1} ) ( {I}_{2} ( {T}_{u} ( p ) ) - {I}_{2} )}{{\{ ( {\sum }_{p ∈ {Ω}_{1 , 2}^{{T}_{u}}} {( {I}_{1} ( p ) - \overline{{I}_{1}} )}^{2} ) ( {\sum }_{p ∈ {Ω}_{1 , 2}^{{T}_{u}}} {( {I}_{2} ( {T}_{u} ( p ) ) - {I}_{2} )}^{2} ) \}}^{\frac{1}{2}}} , \qquad ( 1 )$$

and Ω₁ and Ω₂ represent the subspaces of (Ω⊂′ [symbol] ³) containing the image domains of I₁ and I₂, i.e.

Ω_{1,2}^{T}^{ᵤ}={pεΩ₁|Tᵤ⁻¹(pεΩ₂)}.

We optimized the image similarity measure given by NCC (l_{base}, lₗᵢᵥₑ.) to obtain Tᵤ for each of the 16 images we acquired. We used a local optimization method i.e. Powell's method [16] to optimize the six/twelve dimensional search space that includes three translations, three rotations, and shearing. Powell's method improved the speed of execution and reduced the memory size of the computation, when compared with a gradient-descent-based method during our initial experiments.

### Incremental 2D-3D Registration for Continuous Intra-Biopsy Motion Compensation

The registration to compensate for prostate motion can be performed frequently (e.g., once per second) throughout the biopsy procedure, with the frequency of registration limited by the time required to register a single pair of images. At a given time point denoted by to (time elapsed in seconds from the start of the biopsy), we initialized the source image for the n^{th} registration with the transformation matrix obtained from registrations at previous time points using

Tᵤ=Πₜ₌ₜ_{₀}^{t}^{ₙ}Tᵤ_{ₜ}. (2)

During the n^{th} registration, we found the parameter vector uₜₙ, that gave the optimum NCC measure for the transformation matrix Tᵤₜₙ. We performed the registration for the complete biopsy procedure for the eight subjects described in the previous section using the sequence of live 2D TRUS images recorded every second from the start of the biopsy procedure.

### 2D-3D Registration—Probe Pressure Protocol

3D TRUS images acquired at different probe pressures can provide additional anatomical context to enhance the validation of our registration algorithm. We denote images acquired at low, medium and high probe pressures, respectively, as I_{low} I_{med.}I_{high}.:Ω→ [symbol] We acquired 30 such images from 10 subjects.

We set the image acquired at medium pressure, I_{med}., as the source image. As our target image, we selected 2D slices (I_{(low,high)}) from the 3D images l_{low} and l_{high}. For the 20 registrations performed (using the 30 3D TRUS images) mechanical tracker transformations (T_{Tr}.) were randomly selected from 16 frames (across 8 subjects in the biopsy protocol) occurring an average of 1-2 seconds prior to the firing of the biopsy gun in real biopsy procedures, according to

l_{(low,high)}(p₂)=l_{(low,high)}(T_{Tr}(p₁)) where p₁⊂ψ and p₂⊂Ω.

Hence, the target images are representative of live 2D TRUS images depicting a situation with minimal prostate motion (slice from I_{low}) and substantial prostate motion (slice from I_{high}). Since the physician intentionally applies different levels of pressure during the acquisition, the set of images contains a wide range of prostate displacements and deformations that are intended to represent the extremes of probe pressure during the biopsy procedure to challenge the registration algorithm. For each subject, we perform registration between images I_{med.}-I_{low} and I_{med.}i_{high} by respectively optimizing the image similarity measures, NCC(I_{low},I_{med.}) and NCC(I_{high},I_{med.}) as defined above in Equation 1.

### Validation

### Biopsy Protocol Registration

The registration was validated using manually-identified corresponding intrinsic fiducial pairs (micro-calcifications) [13]. For the images acquired under the biopsy protocol, fiducials appearing in I_{base}, denoted by ƒ_{base}, and the corresponding fiducials from !ₗᵢᵥₑ, denoted by ƒₗᵢᵥₑ, were identified (ƒₗᵢᵥₑ⊂ψ and ƒ_{base} ⊂Ω). We identified 52 fiducial pairs for 16 biopsies in eight patients. These fiducial pairs were used for validation only and were not provided as input to the registration algorithm. The target registration error was calculated as the root mean square (RMS) error

$$\begin{matrix}{TRE}_{b} = \sqrt{\frac{{\sum }_{k = 1}^{{N}_{k}} {( {T}_{Tr}^{- 1} ( {f}_{live}^{k} ) - {T}_{u}^{b} ( {f}_{base}^{k} ) )}^{2}}{{N}_{k}}} , & ( 3 ) \\ {TRE}_{biopsy} = \sqrt{\frac{{\sum }_{b = 1}^{{N}_{b}} {TRE}_{b}^{2}}{{N}_{b}}} & ( 4 )\end{matrix}$$

where N_{b} is the number of biopsies and Nₖ is the number of fiducials identified for a particular pair of images. The TRE was estimated by first calculating RMS values TRE_{b} using the fiducials identified in each pair of images for each biopsy and then calculating the RMS value TRE_{biopsy} for the number of biopsies performed. This approach averaged the contributions to the TRE from the variable number of fiducials manually identified in each pair of images during a biopsy. The TRE before registration was calculated without applying the registration transform Tᵤ in Equation 3 to compare against TRE post registration to assess the improvement.

### Probe Pressure Protocol Registration

In the data set acquired under the probe pressure protocol, full 3D anatomical information for the whole prostate was available for both the source and target images. We manually identified 188 fiducials throughout the 3D volumes obtained from 10 subjects, without limiting the fiducials to lie within the particular extracted plane used in the registration, The TRE was computed as

$$\begin{matrix}{TRE}_{p} = \sqrt{\frac{{\sum }_{k = 1}^{{N}_{k}} {( {T}_{3 D - world} ( {f}_{med}^{k} ) - {T}_{u}^{b} ( {f}_{{( low , high )}^{k}} ) )}^{2}}{{N}_{k}}} , & ( 5 ) \\ {TRE}_{pressure} = \sqrt{\frac{{\sum }_{b = 1}^{{N}_{p}} {TRE}_{p}^{2}}{{N}_{p}}} & ( 6 )\end{matrix}$$

where f{_{med,low,high})⊂. Ω are the fiducials identified in I_{med}, I_{low},I_{high}.

We also computed the optimal rigid alignment using the identified fiducials to define the rigid transformation that yielded the minimum TRE for the given fiducials per patient. To do this, we found the fiducial registration error (FRE) [17] for each set of fiducial pairs in each patient, after transforming the fiducials with the parameters corresponding to the best rigid alignment. With the presence of non-rigid deformations in the probe pressure protocol data set, FRE gives a lower bound on the TREₚᵣₑₛₛᵤᵣₑ that was calculated using a rigid registration.

### GPU Implementation

The step consuming the most computation time during execution of the registration was the calculation of the image similarity metric during optimization, Therefore, we implemented the NCC calculation on an nVidia GTX 690 (Nvidia Corporation, Santa Clara, Calif.) graphics processing unit (GPU) using compute unified device architecture (CUDA), The normalized cross-correlation calculation is inherently parallelizable. Instead of using a sequential approach to transform each voxel independently, we transformed all voxels in the moving image in parallel during each iteration of optimization. These transformations were followed by 3D linear interpolation of image intensities to resample the moving image that was also performed within the GPU. The subsequent calculation of the summations in Equation 1 was also done in parallel with the GPU reduction algorithm to further accelerate the execution. In one iteration of the particle swarm optimization, the calculation for different particles are also madparallel inside the GPU.

### Correlation Between Image Similarity Metric and Misalignment

During registration, we optimized an image similarity metric over a 3D transformation space. The relationship between the image similarity metric and the amount of misalignment not only conveys the suitability of the metric to be used in registration, but also it shows whether the image-similarity metric could be used as an indicator of the misalignment. This may be a useful feature to trigger the registration algorithm in a system that does not continuously compensate for motion as background process during biopsy. To analyze this relationship using the biopsy protocol data, we plotted the calculated normalized cross-correlation measures for each instance before registration, during registration (for each iteration during the optimization) and after registration (after the optimizer converged) and their corresponding TRE_{biopsy} values.

With manually identified fiducials, we should be able to find a plane within the 3D TRUS image that yields near zero TRE. We analyzed the behaviour of normalized cross-correlation near this “optimum” plane by extracting 2D images lying nearby (in terms of the six/twelve parameters, defining 3D translation and rotation) planes in the 3D TRUS image, and computed the image similarity metric for the 2D TRUS image and these nearby 2D images from the 3D TRUS image.

Although this approach does not fully explore the six-dimensional/twelve-dimensional objective function, to simplify the visualization of the results, we analyzed the metrics by varying one degree-of-freedom at a time,

### TRE as a Function of Distance to the Probe Tip

We analyzed the TRE as a function of distance of each fiducial to the ultrasound probe tip, to test if the registration error is larger within the regions of the prostate close to the ultrasound probe. Since we used a rigid/affine transformation during registration, non-rigid deformation of the prostate would be reflected as part of the TRE. Ultrasound probe pressure might cause inconsistent deformation in different regions of the prostate, which could lead to regionally-varying accuracy of motion compensation by a rigid/affine transformation.

### RESULTS

### A. Validation: Biopsy Protocol Data

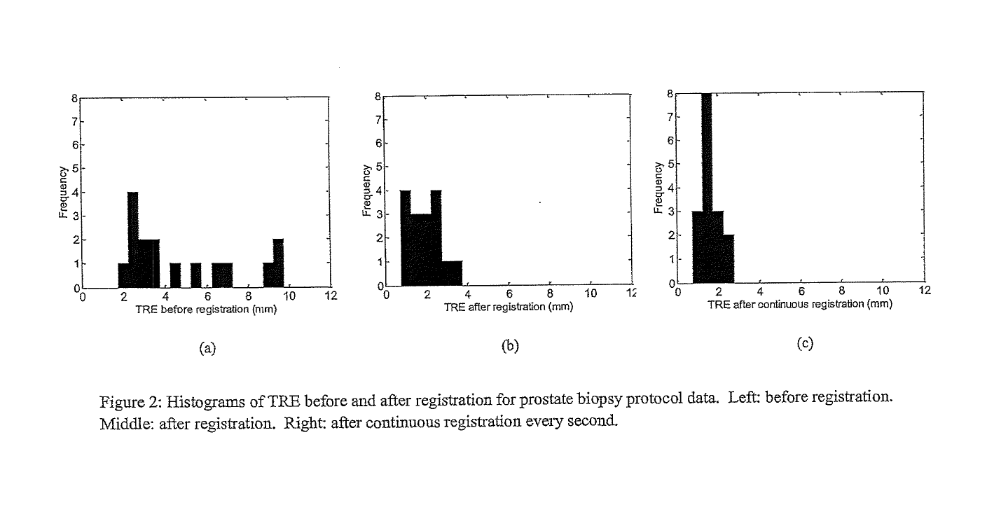

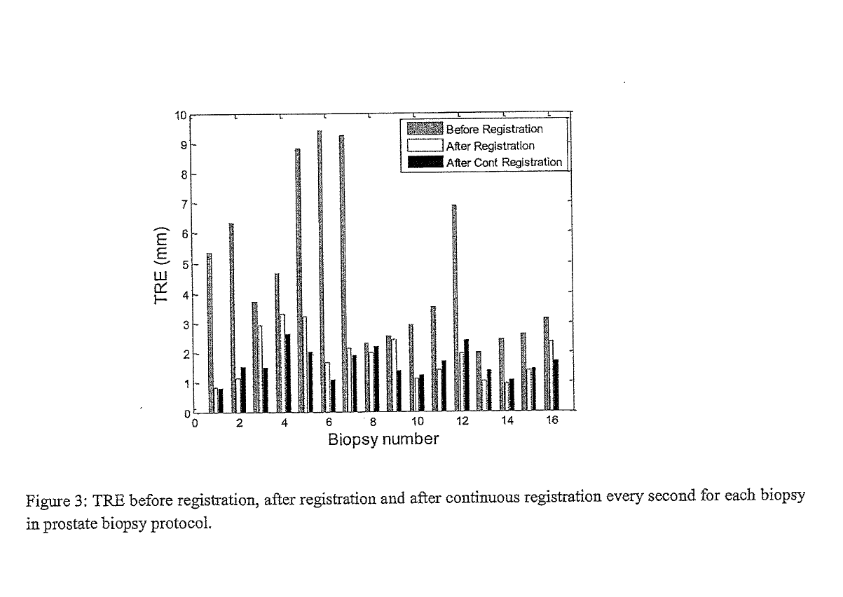

The TRE_{biopsy} was calculated according to Equation 4 and its RMS±std. was found to be 1.87±0.81 mm, after manually localising 52 fiducial pairs over 8 subjects, This was an improvement over 4.75±2.62 mm before registration. Since these TRE distributions were found to be not normally distributed using one-sample Kolmogorov-Smirnov test with a significance level p<0.0001, we tested the null hypothesis that their medians were equal with a non-parametric test using Prism 5.04 (Graphpad Software Inc., San Diego, USA). The Wilcoxon signed rank matched pairs test rejected the null hypothesis (p<0.0001) suggesting that there is a statistically significant difference in TREs before and after registration. When 2D-3D registration was performed incrementally every second during the biopsy, the RMS-std TRE was reduced to 1.63±0.51 mm The mean number of iterations required for convergence decreased from 5.6 to 2.75, FIG. 2 shows changes in TRE distributions before biopsy taken. FIG. 3 shows TRE values for each biopsy.

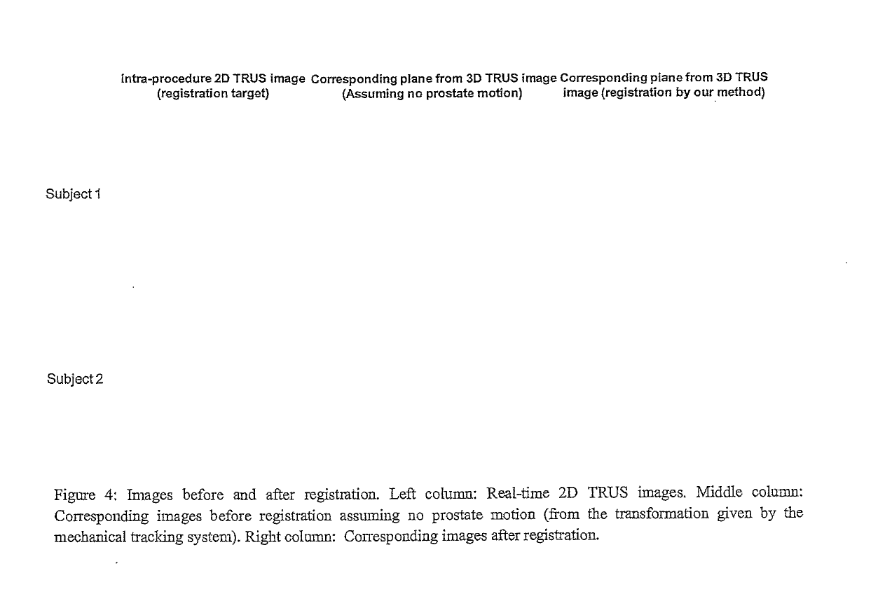

FIG. 4 contains two representative example images, depicting the visual alignment qualitatively. The post-registration TRE of these two example images were found to be 1.5 mm (top row) and 1.2 mm (bottom row), which had improvements from 3.7 mm (top row) and 5.3 mm (bottom row) before registration. Grid lines overlaid at corresponding locations in image space facilitate visual evaluation of the alignment of the anatomy pre- and post-registration.

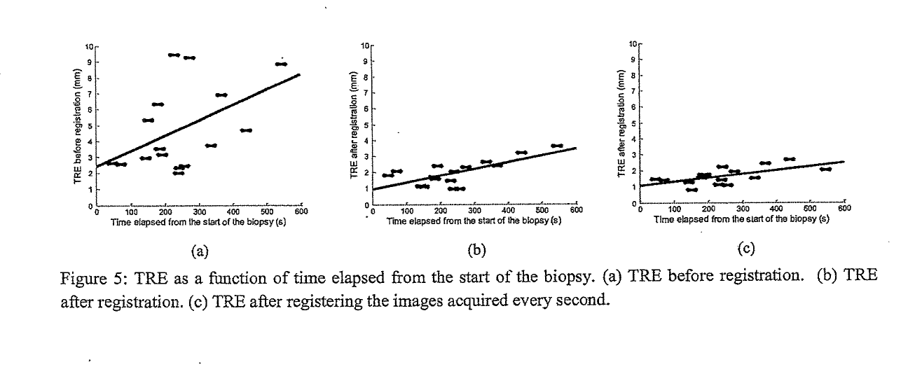

In order to see the effect of patient motion over time during the biopsy session, we analyzed the TREs obtained from eight patients as a function of time elapsed since the start of the biopsy. According to the results shown in FIG. 5, it can be seen that the TRE values before and after registration have an increasing trend with the elapsed time during the biopsy. Weak relationships were found with correlation coefficient (r²) values of 0.23 before registration and 0.41 after registration. When the registration was performed every second, the r² value was found to be 0.37,

### B. Validation: Probe Pressure Protocol Data

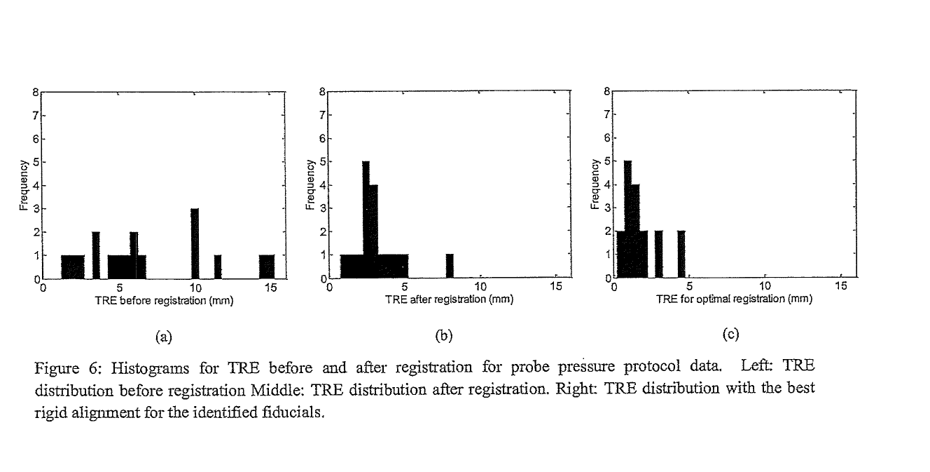

The RMS TRE for the data acquired under the probe pressure protocol was 3.18±1.6 mm, This was an improvement from a 6.89±4.1 mm TRE before registration. Note that we used the fiducials in the whole prostate (not just the slice containing the fiducials) in TRE calculation as given in Equation 6. The mean value for the FRE, corresponding to the best rigid transform that aligns the identified fiducials, was found to be 1.85±1.2 mm. The distribution of TRE values before registration, after registration, and after transforming with the best rigid alignment is shown in FIG. 6. The error in registration includes the errors due to non-rigid deformation occurring within prostate regions outside of the 2D target image (as opposed to the errors arising only due to deformation within the 2D target image as in the biopsy protocol) and the variability in manually locating the fiducials in 3D.

### C. Speed of Execution

After the GPU-accelerated implementation (nVidia GTX 690 GPIJ card and Intel Xeon 2.5 GHz processor) the registration was performed with mean±std times of 1.1±0.1 seconds for the biopsy protocol experiments described herein,

### D. Correlation Between Image Similarity Measure and Misalignment

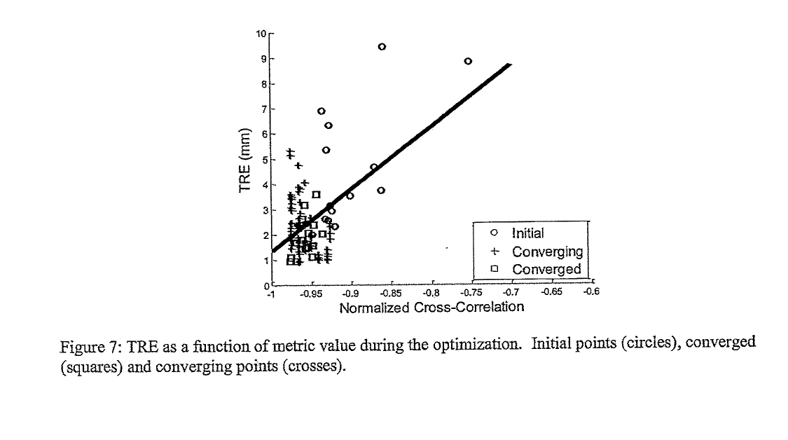

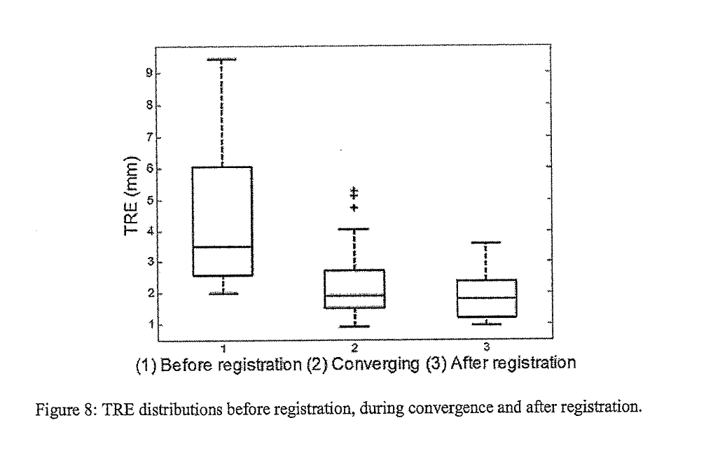

FIG. 7 shows the relationship between the image-similarity measure and values of THE for each transformation obtained during the optimization iterations. The circle points show the values before registration, and the square points show the values after registration converged. The cross points depict the values during convergence. The correlation coefficient (r²), calculated using all points (before, during, and after convergence) in FIG. 7, was found to be 0.23. FIG. 8 shows a box plot of the TRE distributions of the points before registration, during convergence and after registration. While the TRE decreases in general during convergence, a weak correlation can be seen between image similarity measures and TRE from these results,

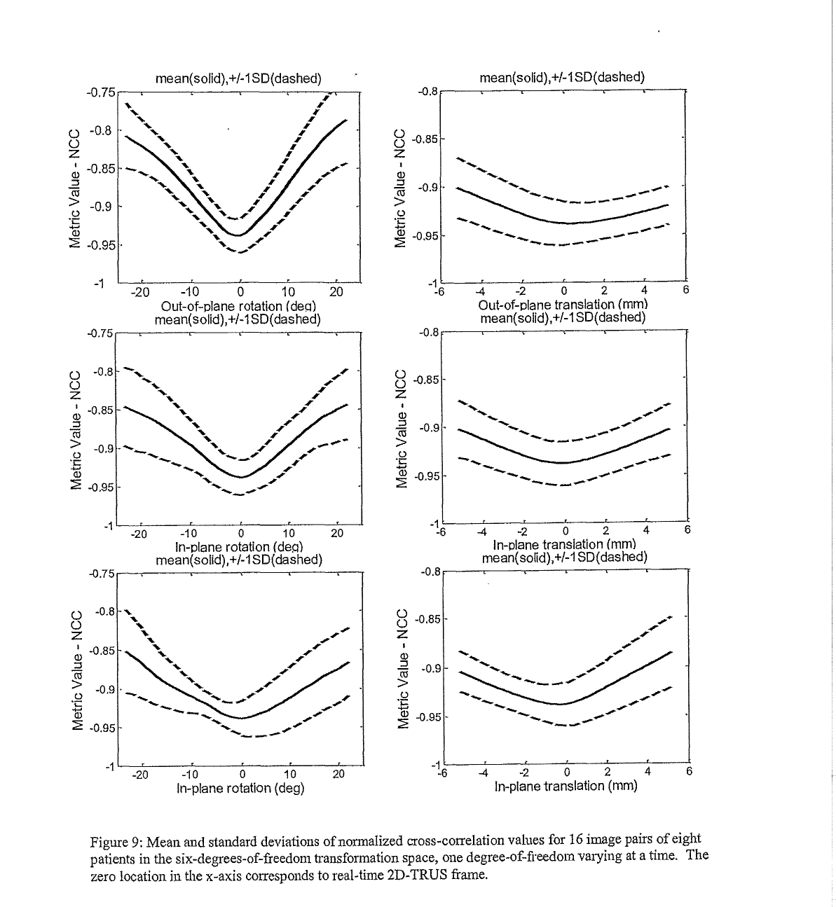

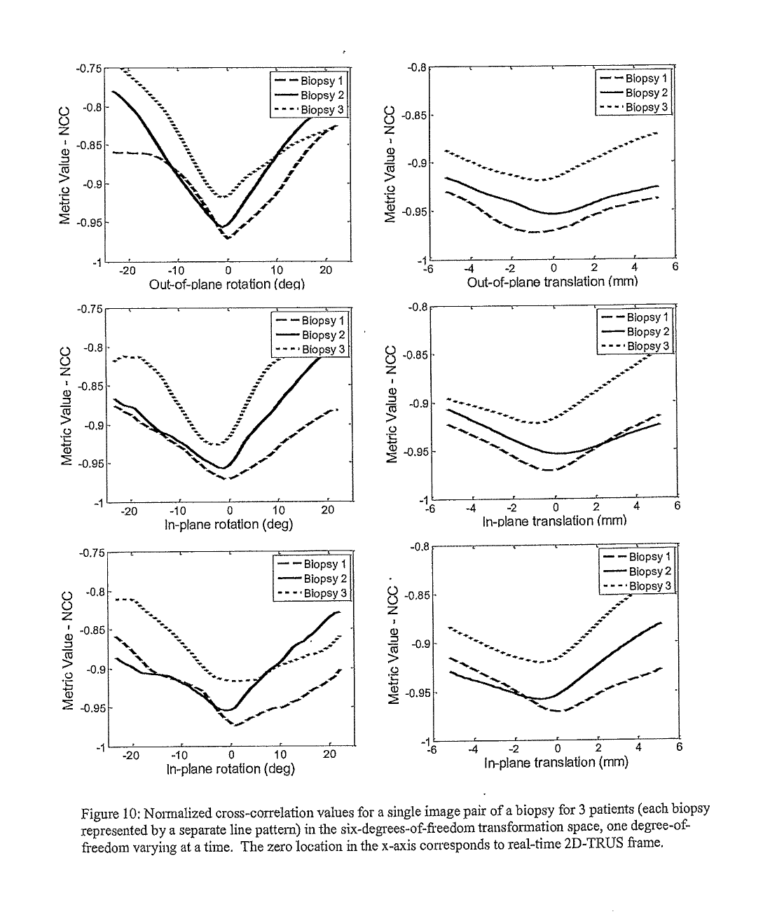

FIG. 9 shows plots of the normalized cross-correlation metric versus out-of-plane, in-plane rotations and translations. The solid curves represent the mean values of the metrics for different out-of-plane rotations and translations for 16 2D TRUS images across eight subjects, and the dashed curves show the values one standard deviation above and below the mean. The convexity of the mean curves gives an indication of the general capture range of the objective functions for many registrations. FIG. 10 shows the three plots of normalized-cross-correlation metrics similarly obtained for a single biopsy in three patients. The generally convex shape of 375 the functions observed in FIG. 9 and FIG. 10 encourages the use of normalized cross-correlation during registration in compensating for prostate motion.

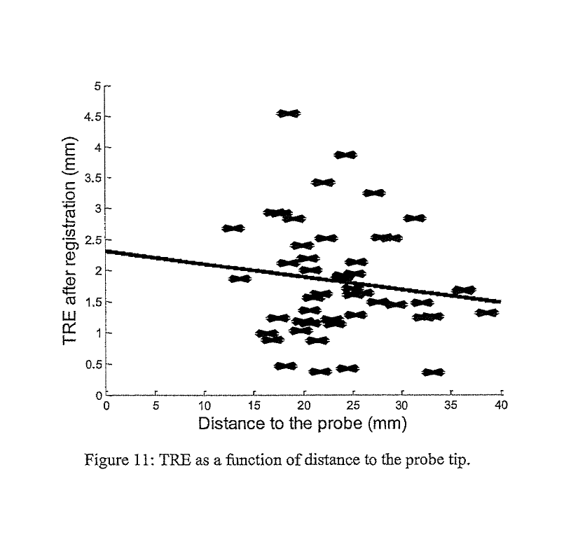

FIG. 11 shows TRE as a function of the distance to the probe tip for each individual. The TRE tends to increase closer to the probe tip (r² value=0.1); however, the correlation between distance to the probe tip and the TRE before registration is weak.

### DISCUSSION AND CONCLUSIONS

### A. Accuracy of Registration

Our image registration method was validated using the fiducials identified in clinical images acquired during the biopsy procedures. There was a significant improvement of TRE after registration in both biopsy and probe pressure protocols. The required accuracy of the biopsy system to guide needles to target locations stems from the size of the smallest clinically-relevant tumours (0.5 cm³, corresponding to a spherical target with 5 mm radius) [18]. A biopsy system with a measured RMS error of 2.5 mm in taking a sample from the intended target will have a probability of at least 95.4% of taking a sample within this 5 mm radius since 5 mm is 2 standard deviations away from the mean of the distribution of targets given by an system with RMS error of 2.5 mm [13]. An image-based registration during the procedure, while compensating for prostate motion, also corrects for tracking errors in the biopsy system, if any. Therefore, if the registration was performed immediately before the physician fires the biopsy gun to capture a tissue sample from the prostate, the targets identified in the pre-acquired 3D image would be aligned with the live 2D TRUS image, with accuracy limited by the TRE of the registration algorithm, However, the motion and deformation induced due to the rapid firing of the biopsy gun, which happens during a sub-second interval remains an error in the biopsy system that is challenging to correct. When targeting a predefined location, the TRE of the motion compensation algorithm and the error during the rapid biopsy-gun firing process, which was quantified in [19], may accumulate and become an important consideration.

Alignment of the targets identified in the 3D TRUS image to the live 2D TRUS image is primarily required immediately before the physician fires the biopsy gun, Consequently, this registration could be integrated into the clinical workflow by executing it just prior to the physician aiming at target locations. However, according to the results, both the accuracy and speed of the registration were improved when the registration was performed on the 2D TRUS images acquired every second, When the baseline 3D TRUS image is updated more frequently, it might improve initialization of 2D TRUS images that follow in subsequent registrations, providing for faster convergence to a suitably accurate optimum. Therefore, in a clinical procedure, this algorithm can be performed in the background continuously compensating for motion.

B, Change of TRE with Time During Biopsy

The weak positive relationship between TRE and time elapsed shown in FIG. 5(a), suggest that the misalignment between pre-acquired and live images increases with time (slope of the best-fit line=9.6 μm/s). After performing the registration just before a biopsy sample is taken, there is still a positive relationship (slope=4.1 □m/s) between TRE and time. This indicates that image pairs, with higher initial misalignments towards the end of the biopsy procedure, were more challenging for the algorithm, In FIG. 5(c), the slope of the best-fit line was lower (slope 2.4 □m/s) when the registrations were performed every second, The improved initializations when performing registrations every second may have induced convergence to a better solution.

### C. Probe Pressure Protocol

In probe pressure protocol, the TRE was 1.2 mm higher than that of the biopsy protocol. This increase could be attributed to the use of fiducials from the whole prostate during validation. The best rigid transform for the selected plane may not necessarily be the best rigid fit for the whole prostate due to non-rigid deformations occurring at different (out of plane) regions of the prostate, Moreover, the high probe pressures intentionally exerted by the physician when acquiring these images might have caused more than the usual deformation that occurs during biopsy. The extreme range of probe pressures and prostate displacement and deformation could make accurate registration more challenging as the algorithm is more susceptible to local minima the further the initialization is from target alignment. However, the fiducial identification process was relatively more straightforward due to the availability of 3D contextual information in both the fixed and moving images,

### D. Correlation Between Similarity Metric and TRE

FIG. 7 shows a weak correlation between similarity metric values before, during and after convergence and the TRE. The generally convex shapes observed in FIG. 9 and FIG. 10 in metric values as a function of different amounts of introduced translations and rotations, suggest that the metric value could be used as a weak indicator to the quality of the registration.

In FIG. 11, a weak negative correlation can be seen between the TRE and distance to the probe tip. This suggests that near the probe tip there could be higher non-rigid deformation of the prostate that may not be accurately compensated with a rigid registration algorithm,

Accurate and quick registration to compensate for motion during biopsy is an important step to improve the accuracy in delivering needle to target locations within the prostate. We presented a 2D-to-3D rigid intensity-based registration algorithm with a measured error of less than 2 mm, validated on clinical human images using intrinsic fiducial markers, to align a 3D TRUS image (with associated prostate biopsy targets) acquired at the start of the procedure to 2D TRUS images taken immediately prior to each biopsy during the procedure. The accuracy and speed of the registration further improves when the baseline 3D image is updated by registering the 2D TRUS images recorded every second during biopsy. Using our high-speed GPU implementation (0.1 seconds total time per registration), this algorithm can be executed in the background during the biopsy procedure in order to align pre-identified 3D biopsy targets with real-time 2D TRUS images. We also presented evidence that image similarity metrics can be used as a weak indicator of the amount of prostate misalignment (with respect to the initially acquired 3D TRUS image), and could be used to trigger the execution of a registration algorithm when necessary.

### Broader Applications

In addition to medical applications, motion compensation has applicability to fields requiring the ability to detect and track moving objects/targets, including machine vision (i.e. dynamic image analysis, object and environment modeling), military, sensor (i.e. object tracking), and mining. Our discovery is intended to be applicable to these applications.

Normally, non-rigid registration is too slow for the calculations described herein. However, we have also discovered that we can make non-rigid registration very fast. See Medical Image Computing and Computer-Assisted Intervention—MICCAI 2013; Computer Science Volume 8149, 2013, pp 195-202 “Efficient Convex Optimization Approach to 3D Non-rigid MR-TRUS Registration”; Yue Sun, et al. It is therefore to be understood that 2D-3D registration can also be non-rigid.

### REFERENCES

1. Canadian Cancer Society's steering committee: Canadian cancer statistics 2012 (2012);
2. Howlader, N., Noone, A. M., Krapcho, M., Neyman, N., Aminou, R., Altekruse, S. F., Kosary, C. L., Ruhl, J, Tatalovich, Z., Cho, H., Mariotto, A., Eisner, M. P., Lewis, D R., Chen, H. S., Feuer, B. J., Cronin, K. A.: SEER Cancer Statistics Review, 1975-2009 (Vintage 2009 Populations), National Cancer Institute. Bethesda, Md., http://seer,cancer.gov/esr/1975_{—}2009_hops09/, based on November 2011 SEER data submission, posted to the SEER web site, April 2012;
3. Leite, K, R, M., Camara-Lopes, L, H, Dall'Oglio, M. F., Cury, J., Antunes, A, A., Safludo, A., Srougi, M.: Upgrading the Gleason score in extended prostate biopsy: Implications for treatment choice. Im, J. Radiat. Oncol., Biol., Phys. 73(2) (2009) 353-356;
4. Bax, J, Cool, D., Gardi, L., Knight, K., Smith, D., Montreuil, J., Sherebrin, S., Romagnali, C., Fenster, A.: Mechanically assisted 3D ultrasound guided prostate biopsy system. Medical Physics 35(12) (2008) 5397-410.
5. Xu, S., Kruecker, J., Turkbey, B., Glossop, N., Singh, A. K., Choyke, P., Pinto, P., Wood, B. J.: Real-time MRI-TRUS fusion for guidance of targeted prostate biopsies. Comput. Aided Surg. 13(5) (2008) 255-264;
6. Cool D., Sherebrin S, Izawa J., Chin J., Fenster, A.: Design and evaluation of a 3D transrectal ultrasound prostate biopsy system. Med, Phys. 35(10) (2008) 4695-4707;
7. Baumann, M., Mozer, P., Daanen, V., Troccaz, J.: Towards 3D ultrasound image based soft tissue tracking: A transrectal ultrasound prostate image alignment system. In: Proceedings of the 10th International Conference on Medical Image Computing and Computer-Assisted Intervention LNCS 4792 (Part II) (2007) 26-33;
8. Baumann, M., Mozer, P., Daanen, V., Troccaz, J.: Prostate biopsy assistance system with gland deformation estimation for enhanced precision. In: Proceedings of the 10th International Conference on Medical Image Computing and Computer-Assisted Intervention LNCS 5761(12) (2009) 67-74.
9. Markelj, P., Tomazevi, D., Likar, B., Pernu§, F.: A review of 3D/2D registration methods for image-guided interventions. Med Image Anal, 16(3) (2010) 642-661;
10. Birkfellner, W, Figl, M., Kettenbach, J., Hummel, J., Homolka, P., Schernthaner, R., Nall, T., Bergmann, H.: Rigid 2D/3D slice-to-volume registration and its application on fluoroscopic CT images. Medical Physics 34(1) (2007) 246-55;
11. Wein, W., Cheng, J. Z, Khamene, A.: Ultrasound based Respiratory Motion Compensation in the Abdomen, MICCAI 2008 Workshop on Image Guidance and Computer Assistance 500 for Soft-Tissue Interventions (2008);
12. Perona, P., Malik, J.: Scale-space and edge detection using anisotropie diffusion. IEEE Transactions on Pattern Analysis Machine Intelligence 12(7) (1990) 629-639;
13. Karnik, V. V., Fenster, A, Bax, J., Cool, D. W., Gardi, L., Gyacskov, I., Romagnoli, C., Ward, A. D,: Assessment of image registration accuracy in three-dimensional transrectal ultrasound guided prostate biopsy, Medical Physics 32(2) (2010) 802-813;
14. Karnik, V. V., Fenster, A, Bax, J, Cool, Romagnoli, C., Ward, A. D.: Evaluation of intersession 3D-TRUS to 3D-TRUS image registration for repeat prostate biopsies. Medical Physics 38(4) (2011) 1832-43;
15, Hajnal, J., Hawkes, D. J., Hill, D.: Medical Image Registration. CRC Press (2001);
16. Press, W. H., Flannery, B, P., Teukolsky, S. A., Vetterling W. T.: Numerical Recipes in C. Cambridge University Press, second edition (1992);
17. Fitzpatrick, J. M., West, J. B., Maurer, Jr., C. R.: Predicting error in rigid-body point-based registration. IEEE Trans. Med. Imaging 17(5) (1998) 694-702;
18. Epstein J. I., Sanderson H., Carter H. B., Scharfstein D.4.: Utility of saturation biopsy to 515 predict insignificant cancer at radical prostectomy. Urology 66(2) (2005) 356-360; and
19. De Silva, T., Fenster, A., Bax, J., Romagnoli, C., Izawa, J., Samarabandu, J., Ward A. D., Quantification of prostate deformation due to needle insertion during TRUS-guided biopsy: comparison of hand-held and mechanically stabilized systems. Medical Physics 38(3) (2011) 1718-31.
20 The ITK Software Guide, Second Edition, Updated for ITK version 2.4
21 Riccardo Poli•James Kennedy•Tim Blackwell, Particle swarm optimization, Swarm Intell (2007) 1: 33-57 DOI 10.1007/s11721-007-0002-0
22. Yukai Hung and WeichungWang, Accelerating parallel particle swarm optimization via GPU, Optimization Methods & Software, Vol. 27, No. 1, February 2012, 33-51.

Although the above description relates to a specific preferred embodiment as presently contemplated by the inventors, it will be understood that the invention in its broad aspect includes mechanical and functional equivalents of the elements described herein.

What is claimed is:

1. A method for generating a motion-corrected 2D image of a target, the method comprising:
acquiring a 3D static image of the target before an imaging procedure;
during the procedure, acquiring and displaying a plurality of 2D real time images of the target;
acquiring one slice of the 3D static image and registering it with at least one 2D real time image;
correcting the location of the 3D static image to be in synchrony with a reference parameter; and
displaying the reference parameter corrected 2D image of the target.

2. The method, according to claim 1, includes: displaying 2D real time images as an ultrasound video stream collected at a video frame rate of up to 30 frames per second.

3. The method, according to claim 1, further comprising: matching and minimizing target goals or metric values for the 2D real time images.

4. The method, according to claim 1, in which the 2D-3D registration is rigid/affine.

5. The method, according to claim 3, in which local optimization searches the minimized value which mature of a 2D slice inside a 3D volume image.

6. The method, according to claim 3, in which global optimization searches the minimized value which mature of a 2D slice inside a 3D volume image.

7. The method, according to claim 3, in which estimated values are estimated from a few prior output parameters of the successful 2D-3D image registrations and the priori from last period of respiration.

8. The method, according to claim 7, in which the estimation can be a polynomial or Fourier series.

9. The method, according to claim 1, in which one slice of the 3D static image is matched to the correct plane as the 2D real time image.

10. The method, according to claim 1, in which the reference parameter is body movement.

11. The method, according to claim 10, in which the 2D real time image is matched according to the body movement.

12. The method, according to claim 1, in which the registering of the 2D and 3D images are done visually.

13. The method, according to claim 1, in which the registering of the 2D and 3D images are done by identifying corresponding points in the 2D and 3D images and finding the best translation/rotation/shearing transform to achieve approximate registration.

14. The method, according to claim 1, in which for each 2D real time image:
determining the corresponding plane in the 3D static image; and
finding the corresponding 2D real time images in the 3D static image volume to determine which slice therein matches the 2D real time image.

15. The method, according to claim 1 further comprising:
minimizing errors or metric values in registering of the 2D and 3D images by applying a local optimization method.

16. The method, according to claim 14, further comprising:
minimizing the errors or metric values in registering of the 2D and 3D images by applying Powell's optimization algorithm.

17. The method, according to claim 14, further comprising:
minimizing the errors or metric values in registering of the 2D and 3D images by applying particle swarm optimization to calculate degree of matching between the 2D and 3D images.

18. The method, according to claim 15, in which Powell's optimization algorithm minimizes registration error measurement by calculating the target registration error (TRE).

19. The method, according to claim 15, in which Powell's optimization algorithm minimizes registration error measurement by calculating the metric value using manually identified fiducials in the target.

20. The method, according to claim 7, in which the multiple initial parameters for 2D-3D image registration include the output parameters of the prior 2D-3D registration; the estimated output parameters using a group of the prior 2D-3D registration; or the output parameter of 2D-3D registration from last period of respiration.

21. The method, according to claim 16, in which the particle swarm optimization increases the registration speed when matching large high-resolution 2D and 3D images comparing with other global optimization method.

22. The method, according to claim 15 or 16, in which Powell's optimization algorithm or the particle swarm optimization is continuously applied throughout the procedure by acquiring and registering the 2D real time images every 30-100 millisecond.

23. The method, according to claim 14 in which if the local optimization method fails, a global optimization method is applied, the global optimization method being particle swarm optimization method.

24. The method, according to claim 12, in which the registration is carried out as a background process to continuously compensate for motion during the procedure.

25. The method, according to claim 1, in which a graphics processing unit (GPU)-accelerates the registration.

26. The method, according to claim 1, in which the target is the liver.

27. The method, according to claim 1, in which the target is the prostate gland.

28. The method, according to claim 1, in which the 2D and 3D images are TRUS images.

29. The method, according to claim 1, in which the imaging procedure is an interventional procedure.

30. The method, according to claim 29, in which the interventional procedure is a biopsy procedure.

31. The method, according to claim 1, in which the imaging procedure is remote sensing (cartography updating),

32. The method, according to claim 1, in which the imaging procedure is astrophotography,

33. The method, according to claim 1, in which the imaging procedure is computer vision in which images must be aligned for quantitative analysis or qualitative comparison.

34. A method for generating a motion-corrected 2D image of a target, the method comprising:
acquiring a 3D static image of the target before an interventional procedure;
during the procedure, acquiring and displaying a plurality of 2D real time images of the target;
acquiring one slice of the 3D static image and registering it with at least one 2D real time image;
correcting the location of the 3D static image to be in synchrony with body motion; and
displaying the motion corrected 2D image of the target.

35. A system for generating a motion-corrected 2D image, the system comprising:
an ultrasound probe for acquiring data from a target during an interventional procedure;
an imaging device connected to the ultrasound probe for displaying data acquired by the ultrasound probe;
a computer readable storage medium connected to the ultrasound probe, the computer readable storage medium having a non-transient memory in which is stored a set of instructions which when executed by a computer cause the computer to:
acquire a 3D static image of the target before the procedure;
during the procedure, acquire and display a plurality of 2D real time images of the target;
acquire one slice of the 3D static image and register it with at least one 2D real time image;
correct the location of the 3D static image to be in synchrony with body motion; and
display the motion corrected 2D image of the target.

36. A system for generating a motion-corrected 2D image, the system comprising:
a probe for acquiring data from a target during an imaging procedure;
an imaging device connected to the probe for displaying data acquired by the probe;
a computer readable storage medium connected to the probe, the computer readable storage medium having a non-transient memory in which is stored a set of instructions which when executed by a computer cause the computer to:
acquire a 3D static image of the target before the procedure;
during the procedure, acquire and display a plurality of 2D real time images of the target;
acquire one slice of the 3D static image and register it with at least one 2D real time image;
correct the location of the 3D static image to be in synchrony with a reference parameter; and
display the reference parameter corrected 2D image of the target.

37. The method, according to claim 1, in which the 2D-3D registration is non-rigid.
